(19) United States
(12) Patent Application Publication
(10) Pub. No.: US 2022/0118199 A1
(43) Pub. Date: Apr. 21, 2022

(54) INHALATION CHAMBER

(71) Applicant: STIPLASTICS, Saint Marcellin (FR)

(72) Inventors: Etienne Brichet-Billet, Notre Dame De L'osier (FR); Guillaume Zoczek Tullins, Tullins (FR); Ludovic Soual, Romans Sur Isere (FR); Hugues Brouard, Rochefort Samson (FR)

(21) Appl. No.: 17/422,663

(22) PCT Filed: Jan. 14, 2020
(86) PCT No.: PCT/FR2020/050041

§ 371 Date: Jul. 13, 2021

(30) Foreign Application Priority Data
Jan. 14, 2019 (FR) 1900302

(51) Int. Cl. A61M 15/00; A61M 16/20; A61M 16/04

(52) U.S. Cl. CPC A61M 15/0088; A61M 15/0065; A61M 15/0016; A61M 16/208; A61M 2205/215; A61M 2205/15; A61M 2205/581; A61M 2205/582; A61M 16/0488

(57) ABSTRACT

The present invention relates to an inhalation chamber including a body having an inlet orifice suitable for being connected to the outlet end piece of a metered dose inhaler and an outlet orifice suitable for being connected to a mouthpiece or a mask to apply on the face of a patient, and a support suitable for receiving the outlet end piece of the metered dose inhaler, the support opening into the inlet orifice, characterised in that the axis (X) of the inlet orifice is non-colinear with the axis (Y) of the outlet orifice and in that the support is pivotally mounted on the body around the axis (X) of the inlet orifice.

### CROSS-REFERENCE TO RELATED APPLICATIONS

This application is a U.S. National stage of International Patent Application No. PCT/FR2020/050041 filed Jan. 14, 2020, which claims the benefit of priority of French Patent Application No. 1900302 filed Jan. 14, 2019, the respective disclosures of which are each incorporated herein by reference in their entireties.

### FIELD OF THE INVENTION

The invention relates to an inhalation chamber.

### BACKGROUND

Certain medicines, for example medicines aiming to treat asthma, are designed to be administered in the form of microparticles in the lungs of the patient.

The medicine is packaged in a metered dose inhaler (also known by the acronym MDI) which, under the effect of pressure on an actuator exerted by a user (the patient himself or a third party, notably in the case where the patient is a child), expulses a dose of medicine in the form of microparticles at high speed (of the order of 100 km/h).

Due to the difficulty that there may be in perfectly synchronising actuation of the metered dose inhaler and oral aspiration, and so that the medicine is not deposited in the oral cavity of the patient but reaches the pulmonary alveoli, it is known to use an inhalation chamber which is a device defining a closed volume comprising an inlet orifice into which opens the outlet end piece of the metered dose inhaler and an outlet orifice in fluidic connection with a mouthpiece or a mask applied on the face of the patient. The microparticles are suspended in this closed volume, which enables the patient to inhale them by simply breathing through the outlet orifice.

Known inhalation chambers have a tubular shape, the inlet orifice and the outlet orifice being arranged at the two ends of the tube.

However, the handling of the inhalation chamber and the application of the mouthpiece or the mask on the face of the patient are relatively difficult.

In fact, due to the length of the tube, an important distance exists between the metered dose inhaler and the face of the patient. The patient or the user must thus hold in one hand the mouthpiece and in the other hand the actuator of the metered dose inhaler. This is particularly impractical when the patient is a child; in fact, it is often necessary that the user places a hand behind the head of the child in order to reassure him and/or to maintain his head during the application of the mask. The other hand, which manipulates the actuator of the inhaler, is then particularly far from the first one, which makes the correct maintaining of the mask on the face difficult.

Furthermore, it is recommended to maintain the metered dose inhaler oriented vertically, with the microparticles outlet end piece situated at the bottom and the end of the actuator situated at the top. In the remainder of the text this position of the inhaler is designated “vertical position”.

Yet, in certain cases, this orientation may be difficult to conserve. For example, when the patient is lying down, it is generally not possible to maintain the inhaler vertical during the administration of the medicine.

### DESCRIPTION OF THE INVENTION

An aim of the invention is to design an inhalation chamber that is more ergonomic and making it possible to use the metered dose inhaler as best recommended, namely in vertical position, whatever the condition/position of the patient.

To this end, the invention proposes an inhalation chamber comprising a body having an inlet orifice suitable for being connected to the outlet end piece of a metered dose inhaler and an outlet orifice suitable for being connected to a mouthpiece or a mask to apply on the face of a patient, and a support suitable for receiving the outlet end piece of the metered dose inhaler, said support opening into the inlet orifice, characterised in that the axis of the inlet orifice is non-colinear with the axis of the outlet orifice and in that said support is pivotally mounted on the body around the axis of the inlet orifice.

According to a preferred embodiment, the pivot axis of the support and the axis of the outlet orifice are orthogonal.

In a particularly advantageous manner, the body comprises two dismantlable shells.

Preferably, at least one of said shells is transparent.

According to an embodiment, the support comprises an elastomeric portion suitable for ensuring a leak tight connection between the end piece of the metered dose inhaler and the support.

According to an embodiment, the inhalation chamber further comprises a sensor configured to detect the inclination of the metered dose inhaler.

Said sensor is advantageously suitable for being coupled to a control unit configured to compare the measured inclination with an acceptable inclination range and to emit a signal depending on the result of said comparison destined for a luminous indicator, an audible warning device and/or a vibrator.

According to an embodiment, the body comprises at least one luminous indicator configured to light up differently according to said comparison.

In a particularly advantageous manner, the body further comprises two arrow shaped luminous indicators arranged on either side of the support to indicate two opposite directions of rotation of said support, the control unit being configured to command the lighting of one of said indicators along the direction of rotation to apply to the support to incline the metered dose inhaler according to the acceptable inclination range.

The inhalation chamber may further comprise a mask rotationally mounted on the body in aeraulic connection with the outlet orifice.

### BRIEF DESCRIPTION OF THE FIGURES

Other characteristics and advantages of the invention will become clear from the detailed description that follows, with reference to the appended drawings in which:

FIG. 1 is a perspective view of an inhalation chamber according to a first embodiment;

FIG. 2 is another perspective view of the inhalation chamber of FIG. 1;

FIG. 3 is a side view of the inhalation chamber of FIG. 1 with the metered dose inhaler in vertical position;

FIG. 4 is a side view of the inhalation chamber of FIG. 1 with a first other orientation of the inhalation chamber, the metered dose inhaler still being in vertical position;

FIG. 5 is a side view of the inhalation chamber of FIG. 1 with a second other orientation of the inhalation chamber, the metered dose inhaler still being in vertical position;

FIG. 6 shows a perspective view and a top view of an inhalation chamber according to a second embodiment;

FIG. 7 shows a perspective view and a top view of an inhalation chamber according to a third embodiment;

FIG. 8 shows a side view of a metered dose inhaler.

Identical reference signs from one figure to the other designate identical elements or at least elements fulfilling the same function.

### Detailed Description of Embodiments of the Invention

A user and a patient are mentioned in the present text. The user is a person who manipulates the inhalation chamber and who actuates the metered dose inhaler; the patient is a person who inhales the medicine through the inhalation chamber. The patient and the user may naturally be the same person, notably in the case of an adult patient able to self-administer a dose of medicine. On the other hand, in the case of a child or an adult who is not able to manipulate the inhalation chamber himself, it is a third party that manipulates the inhalation chamber.

In a manner known per se, the metered dose inhaler comprises a body and an actuator slidably moveable in the body facing a metering valve. The actuator is in the form of a tank containing a propellant gas and a micronized suspension of the medicine. By pressing on the actuator, a user releases a determined dose of medicine through the metering valve. The metering valve opens into an outlet end piece of the body, the axis of the outlet end piece being non-colinear with the sliding axis of the actuator. Preferably, the sliding axis of the actuator must be maintained vertical during use of the metered dose inhaler.

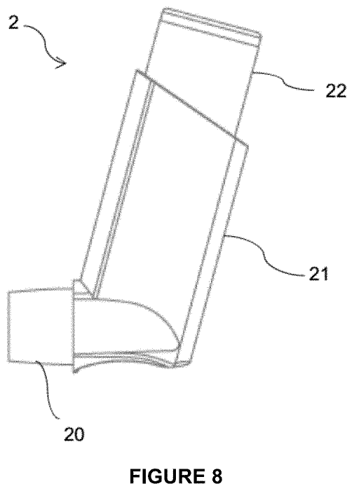

FIG. 8 shows a side view of such a metered dose inhaler 2, comprising a body 21 and an actuator 22, which contains the propellant gas and the microparticles of medicine, slidingly mounted in the body 21. The body comprises a microparticles outlet end piece 20 which is inclined with respect to the sliding axis of the actuator 22.

Depending on the manufacturer, the shape of the metered dose inhaler, notably the shape and the dimensions of the end piece, is liable to vary. However, advantageously, the inhalation chamber is not limited to a specific metered dose inhaler but, as explained below, can receive different commercially available metered dose inhalers.

The inhalation chamber comprises a body having an inlet orifice suitable for being connected to the outlet end piece of a metered dose inhaler and an outlet orifice suitable for being connected to a mouthpiece or a mask to apply on the face of a patient.

According to an embodiment, the mouthpiece is integral with the body (it may be for example moulded with the body) and the mask is suitable for being removably mounted on the body. Thus, the body may be used either by a child patient (with the mask) or by an adult patient (directly with the mouthpiece). The mask comprises a metering valve through which passes the air breathed out by the patient.

The outlet orifice is provided with a volume valve which avoids any aeraulic return towards the inside of the body. A duckbill valve is particularly preferred on account of its capacity to open up widely during an aspiration without excessive effort by the patient, then to close in a leak tight manner, so as to avoid any entry of air into the body during an expiration.

The volume valve and the metering valve may advantageously be made of medical grade silicone.

Advantageously, the mask is pivotally mounted on the mouthpiece, so as to make it possible to adjust the orientation of the mask with respect to the body, notably as a function of the position of the patient.

The body defines, between the inlet orifice and the outlet orifice, a sufficiently large closed volume to enable the suspension of the particles. The shape of this closed volume is notably defined as a function of the location of the inlet and outlet orifices to minimise the presence of zones in which the suspended medicine could be liable to be retained or deposited.

The metered dose inhaler is mounted on the body by means of a support which opens into the inlet orifice, the outlet end piece of the metered dose inhaler being in aeraulic connection with the inlet orifice of the body to enable the introduction of the microparticles of the metered dose inhaler into the body.

Unlike known inhalation chambers, the inhaler support is not fixed with respect to the body, but pivoting with respect to the body, and is so around the axis of the inlet orifice, which is non-colinear with the axis of the outlet orifice. Axis of the outlet orifice is taken to mean an axis perpendicular to the plane formed by the outlet orifice.

The inner shape of the support is advantageously adaptable to the shape of the outlet end piece of the metered dose inhaler, which is generally circular or oblong, in such a way as to ensure leak tightness between the support and the end piece. According to a preferred embodiment, the support comprises an elastomeric portion deformable according to the shape of the end piece of the metered dose inhaler, in order to ensure both the leak tightness and the mechanical strength of the connection. The support may be produced by injection of two different plastic materials, a rigid material ensuring the connection with the body and an elastomeric material ensuring the connection with the end piece of the metered dose inhaler.

The pivot axis being non-colinear with the axis of the outlet orifice, it is possible by a simple pivoting of the support to ensure that the metered dose inhaler remains oriented vertically, whatever the position of the patient.

According to a preferred embodiment, the pivot axis is orthogonal to the axis of the outlet orifice.

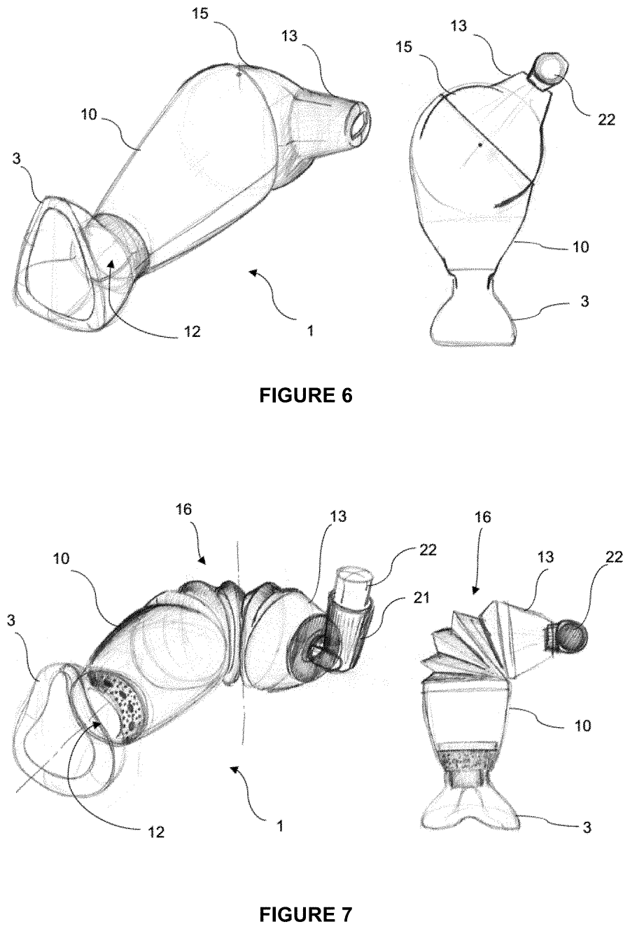

The movement of the support with respect to the body may not be limited to a rotation around a single axis. Optionally, an additional degree of rotational freedom may be provided by a swivel joint (cf. FIG. 6) or a gusset (cf. FIG. 7).

By being arranged on one side of the body, laterally with respect to the mouthpiece or to the mask, the support forms a handle easy to manipulate by the user. The external shape of the support may be defined to improve the ergonomics of the handle.

In general, the user holds the inhalation chamber at the level of the support/handle by the hand that he uses preferentially in his everyday tasks (his right hand if he is right-handed, his left hand if he is left-handed). The support may thus be pivoted so as to be able to be held by the right hand or the left hand of the patient, while conserving the metered dose inhaler vertical in both cases.

Furthermore, the position of the support with respect to the outlet orifice may be modified depending on whether the user is the patient or a third party. Indeed, in the latter case, the user generally places himself facing the patient, such that the handling of the inhalation chamber is inversed.

The pivoting of the support makes it possible to place the metered dose inhaler on the preferred side for the user, whether it is the patient or a third party, while maintaining the metered dose inhaler oriented vertically with the outlet end piece in the lower part.

Said support/handle is also closer to the face of the patient than in inhalation chambers of the prior art. It thus allows one-handed use of the inhalation chamber (the user holding the inhalation chamber by the support/handle is able to apply correctly the mouthpiece or the mask on the face of the patient while actuating the actuator). The other hand thus remains free, for example to reassure the patient when said patient is a child.

Another advantage of the positioning of the support of the inhaler around an axis non-colinear with that of the axis of the outlet orifice is to impose on the flow of microparticles an L-shaped trajectory inside the body. This trajectory has the effect of slowing down the microparticles and enabling the largest microparticles (which must not be inhaled by the patient) to bump onto the face of the body opposite to the inlet orifice and to fall by gravity to the bottom of the body instead of coming out through the outlet orifice.

The body is manufactured from antistatic materials. In a particularly advantageous manner, at least one of these materials is a transparent thermoplastic material, such as ABS (acrylonitrile butadiene styrene) for example. Thus, the user can visualise the microparticles inside the body and observe the operation of the volume valve.

According to a preferred embodiment, the body is formed of two shells assembled in a dismantlable manner by any suitable means (for example by interlocking, snap fitting, etc.) enabling manual dismantling by the user. This makes it possible to clean and dry the inside of the body easily after use of the inhalation chamber.

Advantageously, said shells are assembled along a plane that contains the pivot axis of the support of the metered dose inhaler. The shell that contains the outlet orifice may be made of an opaque antistatic material, whereas the opposite shell may be made of a transparent antistatic material. Grades of ABS suitable for these two functions exist.

According to an embodiment, the support of the metered dose inhaler is provided with an inclination sensor suitable for measuring the inclination of the sliding axis of the actuator with respect to the vertical. Said sensor is coupled to a control unit which makes it possible to compare the measured inclination with a range of acceptable inclination values around the vertical.

The control unit may be coupled to a luminous indicator, an audible warning device and/or a vibrator, for example arranged on the body, activated when the measured inclination is outside of the aforementioned range.

According to an embodiment, the body comprises three luminous indicators, a main indicator being configured to be lit up with a green colour if the inclination of the metered dose inhaler is correct, and with a red colour in the case of incorrect inclination. The two other indicators are in the form of two arrows arranged on either side of the support and each indicating a different direction of rotation around the pivot axis of the support. If the main indicator is on red, one of the arrows is lit up to indicate in which direction to turn the support to obtain a correct inclination of the metered dose inhaler, which, once the movement has been made, makes the main indicator switch to green.

Alternatively, the control unit may communicate with an electronic device provided with a screen, such as a mobile telephone, so as to display on the screen the inclination of the metered dose inhaler with respect to the vertical. Arrows may also be displayed on the screen to procure for the user indications to correct a possible incorrect inclination.

According to an embodiment, the inhalation chamber also comprises a device suitable for measuring the speed and/or the flow rate of air breathed in by the patient, in such a way as to be able to carry out a monitoring of the improvement or the deterioration of the respiratory condition of the patient.

For example, said device may comprise an accessory which creates a closed cavity around the metered dose inhaler support, the air inlet of which is equipped with a means for measuring speed/flow rate of inhaled air. This solution makes it possible to avoid hindering the route of the medicine between the metered dose inhaler and the mouth of the patient.

A similar solution could be used to measure the expiration speed and/or flow rate of the patient, above the metering valve.

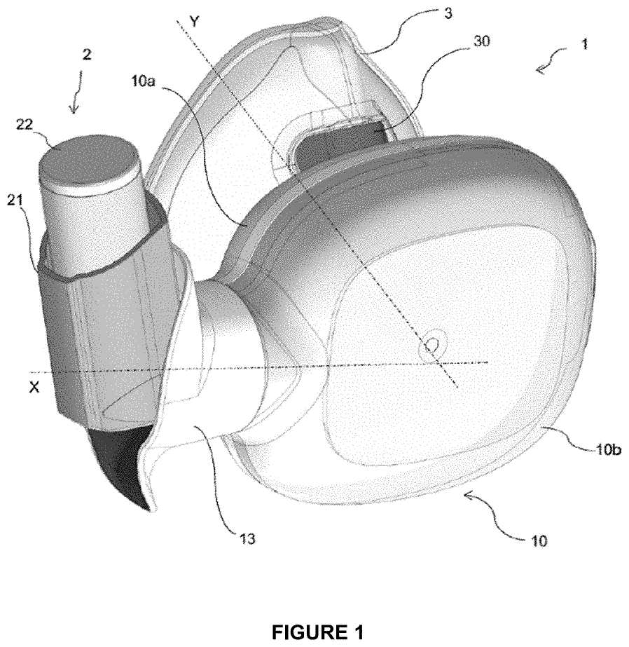

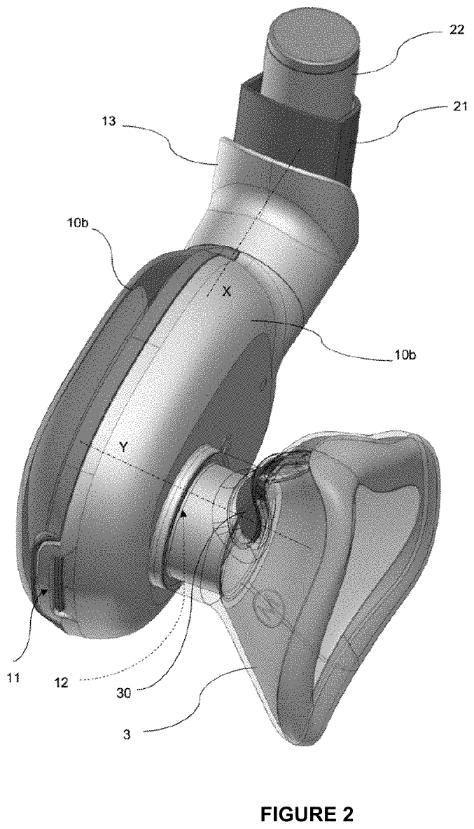

FIGS. 1 to 5 illustrate a first embodiment of the inhalation chamber.

The chamber 1 comprises a body 10, a support 13 suitable for receiving the outlet end piece of the metered dose inhaler 2, opening into the body, and a mask 3 in aeraulic connection with an outlet orifice 12 of the body. The axes X and Y are respectively the pivot axis of the support 13 with respect to the body and the axis of the outlet orifice 12 of the body.

In a manner known per se, a volume valve (not visible in the figures), for example of duckbill type, is arranged in the outlet orifice. The mask furthermore comprises a metering valve 30.

Due to the position of the support with respect to the body, said axes are not colinear and are advantageously orthogonal.

The body 10 comprises two shells 10a, 10b assembled along a joint plane which contains the pivot axis of the support 13. Preferably, the shell 10a, which comprises the outlet orifice, is made of an opaque plastic material, such as antistatic ABS, whereas the shell 10b is made of a transparent plastic material, such as antistatic ABS. The shells 10a, 10b comprise on their periphery complementary shapes enabling a leak tight fitting together and a maintaining by friction, for example a snap fitting closing system 11. They may thus be easily separated by a tractive force of the user in a direction perpendicular to the joint plane with a view to be cleaned then re-assembled.

Each of the shells comprises half of a cylindrical sleeve. After assembly of the shells, the support 13 is tightly press fitted onto said sleeve. The surfaces opposite the sleeve and the support are designed to avoid unwanted dismantling of the support 13 (while allowing its dismantling by application of a sufficient force by the user) while allowing a rotation of the support around the sleeve. Said rotation may be free or potentially indexed by a snap fitting system.

The support 13 comprises on its lower face intended to receive the end piece of the metered dose inhaler an elastomeric portion 14 which makes it possible to ensure a leak tight connection with the end piece while adapting to the shape thereof.

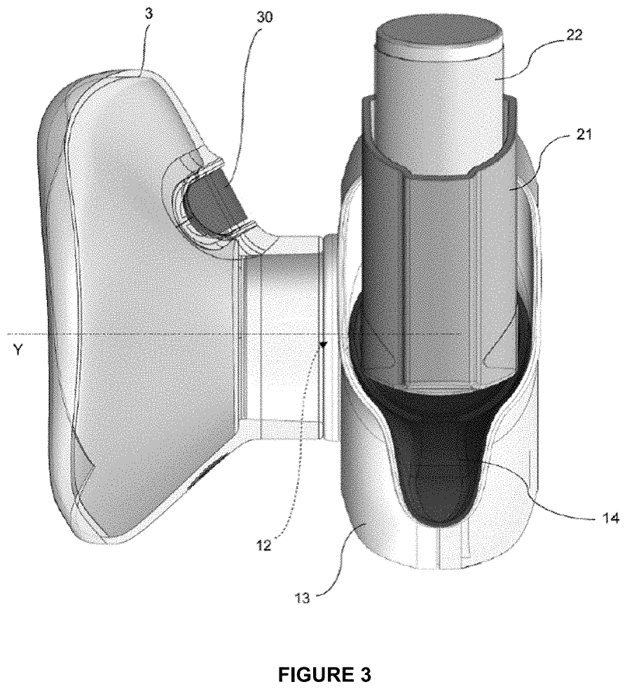

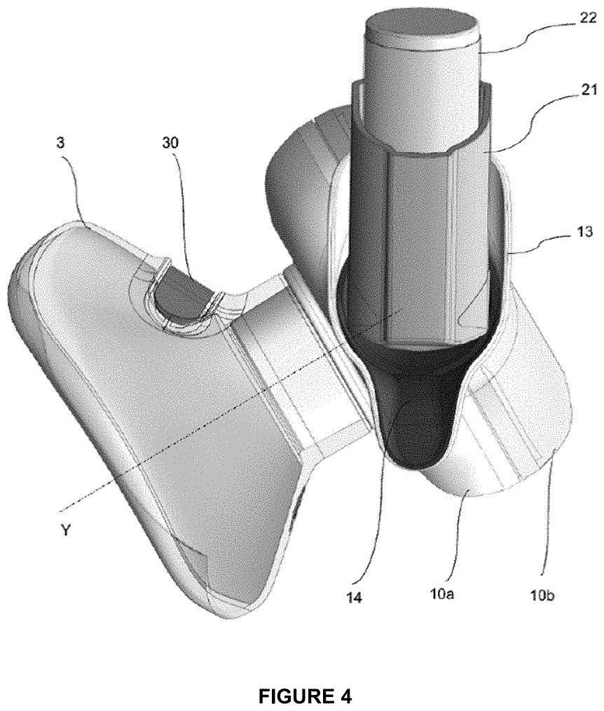

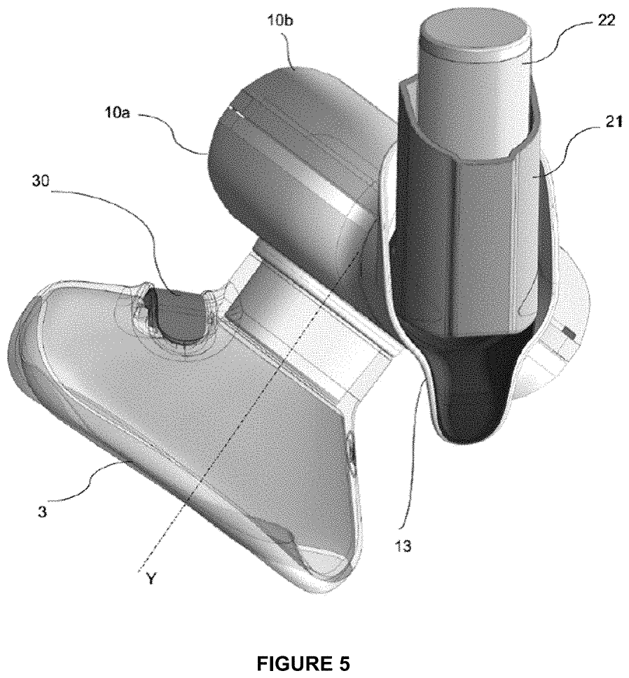

In FIG. 3, the inhalation chamber is shown in a position suitable for application on a seated or standing patient; FIGS. 4 and 5 show the same inhalation chamber in inclined positions suitable for a half-seated patient. As may be seen, the pivoting of the support makes it possible to conserve a vertical orientation of the metered dose inhaler. In FIGS. 3 to 6, the pivot axis of the support 13 is perpendicular to the plane of the sheet.

FIG. 6 illustrates a second embodiment of the inhalation chamber.

Compared to the preceding embodiment, the body 10 has a lengthened shape with the outlet orifice 12 at a first end and a swivel joint 15 at a second end opposite to the first to couple the support 13 for the metered dose inhaler to the body 10. Said swivel joint thus provides an additional degree of freedom to orient the support with respect to the body. Thanks to this mechanism, the metered dose inhaler may be maintained in vertical position whatever the position of the patient.

In this configuration, the body 10 may be made in one piece by moulding of an antistatic plastic material, preferably transparent. The swivel joint is next inserted into the second end of the body 10, preferably in a removable manner in order to enable the cleaning and the drying of the inhalation chamber.

Depending on the applications, a mask 3 may be reversibly mounted on the first end of the body 10.

FIG. 7 illustrates a third embodiment of the inhalation chamber.

The construction of the body 10 is substantially identical to that of the second embodiment.

In this alternative, the support 13 of the metered dose inhaler is coupled to the body by means of a gusset 16 which may be deformed along at least two axes.

In a particularly advantageous manner, the gusset 16 may be dismantled from the body in order to enable the cleaning and the drying of the inhalation chamber.

1. An inhalation chamber comprising
a body having an inlet orifice suitable for being connected to an outlet end piece of a metered dose inhaler and an outlet orifice suitable for being connected to a mouthpiece or a mask to apply on a face of a patient, and
a support suitable for receiving the outlet end piece of the metered dose inhaler, said support opening into the inlet orifice,
wherein an axis of the inlet orifice is non-colinear with an axis of the outlet orifice and the support is pivotally mounted on the body around the axis of the inlet orifice.

2. The inhalation chamber of claim 1, wherein a pivot axis of the support and the axis of the outlet orifice are orthogonal.

3. The inhalation chamber of claim 1, wherein the body comprises two dismantlable shells.

4. The inhalation chamber of claim 3, wherein at least one of said shells is transparent.

5. The inhalation chamber of claim 1, wherein the support comprises an elastomeric portion configured to ensure a leak tight connection between the end piece of the metered dose inhaler and the support.

6. The inhalation chamber of claim 1, further comprising a sensor configured to detect the inclination of the metered dose inhaler.

7. The inhalation chamber of claim 6, wherein said sensor is configured to be coupled to a control unit configured to compare the measured inclination with an acceptable inclination range and to emit a signal depending on the result of said comparison destined for a luminous indicator, an audible warning device and/or a vibrator.

8. The inhalation chamber of claim 7, wherein the body comprises at least one luminous indicator configured to light up differently depending on said comparison.

9. The inhalation chamber of claim 8, wherein the body further comprises two arrow shaped luminous indicators arranged on either side of the support to indicate two opposite directions of rotation of said support, the control unit being configured to command the lighting up of one of said indicators along the direction of rotation to apply to the support to incline the metered dose inhaler according to the acceptable inclination range.

10. The inhalation chamber of claim 1, further comprising a mask rotationally mounted on the body in aeraulic connection with the outlet orifice.
